(19) United States
(12) Patent Application Publication
(10) Pub. No.: US 2005/0249820 A1
(43) Pub. Date: Nov. 10, 2005

(54) Nutritional supplement for the treatment and prevention of macular degeneration

(75) Inventor: Abu Alam, Lake Forest, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Akorn, Inc., Buffalo Grove, IL (US)

(21) Appl. No.: 10/838,467

(22) Filed: May 4, 2004

(51) Int. Cl. A61K 33/32; A61K 35/78; A61K 31/12; A61K 31/355; A61K 31/375

(52) U.S. Cl. 424/638; 424/643; 514/474; 514/458; 514/690; 514/725

(57) ABSTRACT

The invention provides a nutritional supplement in the form of one or more capsules comprising Vitamin A, Vitamin C, Vitamin E, and at least one carotenoid. The invention also provides a nutritional supplement in the form of one or more capsules comprising Vitamin A, Vitamin C, Vitamin E, zinc, copper, and at least one carotenoid. The invention further provides a method for the treatment and/or prevention of macular degeneration comprising the step of orally administering to a patient the nutritional supplement of the invention.

### FIELD OF THE INVENTION

This invention pertains to a nutritional or dietary supplement comprising Vitamin A, Vitamin C, Vitamin E, and at least one carotenoid. The nutritional or dietary supplement is intended to strengthen and promote retinal health through the prevention, stabilization, reversal and/or treatment of visual acuity loss in people with particular ocular diseases.

### BACKGROUND OF THE INVENTION

Macular degeneration associated with age and drusen is the leading cause of severe visual acuity loss in the United States and Western Europe in persons aged 55 years or older. Age-related macular degeneration (AMD) is a collection of clinically recognizable ocular findings that can lead to blindness. The findings include drusen, retinal pigment epithelial (RPE) disturbance—including pigment clumping and/or dropout, RPE detachment, geographic atrophy, subretinal neovascularization and disciform scar. Not all these manifestations are needed for AMD to be considered present. The prevalence of persons with ophthalmoscopically or photographically identifiable drusen increases with age, and most definitions of AMD include drusen as a requisite. However, drusen alone do not seem to be directly associated with vision loss. It is rather, the association of drusen with the vision-threatening lesions of AMD (i.e., geographic atrophy, RPE detachment and subretinal neovascularization) that has led to their inclusion in the definition of AMD. Although recent studies have demonstrated the benefit of laser photocoagulation in those individuals with macular degeneration who develop acute, extrafoveal choroidal neovascularization, no treatment has been shown to be of benefit to the majority of people who have AMD. The cause of macular degeneration is unknown.

Toxicity from free radicals and oxidizers has generated significant interest with regard to macular degeneration and the progression thereof. Circumstantial evidence indicates that protection against phototoxicity and oxidizers, such as would be provided by antioxidants, could slow the onset and progression of age-related macular degeneration as well as cataracts.

Attention has also been focused on the possible involvement of various minerals in retinal disease. For example, zinc has received particular notice in this regard due to the observation of high concentrations of zinc in ocular tissues, particularly the retina, pigment epithelium and choroid. Zinc is an important micronutrient that plays an essential role in human growth and function. Zinc is necessary for the activity of over a hundred enzymes, including carbonic anhydrase, superoxide dismutase and alkaline phosphatase. Zinc acts as a cofactor for numerous metalloenzymes, including retinol dehydrogenase and catalase. Zinc also is a cofactor in the synthesis of extracellular matrix molecules, is essential for cell membrane stability, is needed for normal immune function, is associated with melanin, and is taken up in a facilitated manner by the retinal pigment epithelium. While many existing nutritional supplements seek to supplement mineral (e.g., zinc, copper, etc.) intake, these nutritional supplements often fail to account for the varying needs of the patient for mineral supplementation and the possible negative impact on the patient's health resulting from the administration of unnecessarily high amounts of minerals.

Accordingly, a need remains in the art for a composition and method for the treatment of visual acuity loss due to ocular diseases, such as macular degeneration or cataracts. The invention provides such a composition and method. These and other advantages of the invention, as well as additional inventive features, will be apparent from the description of the invention provided herein.

### BRIEF SUMMARY OF THE INVENTION

In one embodiment, a nutritional supplement of the present invention comprises, on a 24-hour dosage basis: (a) about 500% to about 1200% of the RDA of Vitamin A, (b) about 700% to about 900% of the RDA of Vitamin C, (c) about 1300% to about 2800% of the RDA of Vitamin E, (d) less than about 5% of the RDA of zinc, (e) less than about 5% of the RDA of copper, and (f) about 5 mg to about 35 mg of at least one carotenoid, wherein the at least one carotenoid is not β-carotene, and wherein the nutritional supplement is provided in the form of one or more capsules.

In a second embodiment, a nutritional supplement of the present invention comprises, on a 24-hour dosage basis: (a) about 25,000 IU to about 60,000 IU of Vitamin A, (b) about 420 mg to about 540 mg of Vitamin C, (c) about 390 mg to about 840 IU of Vitamin E, (d) less than about 0.75 mg of zinc, (e) less than about 0.1 mg of copper, and (f) about 5 mg to about 35 mg of at least one carotenoid, wherein the at least one carotenoid is not β-carotene, and wherein the nutritional supplement is provided in the form of one or more capsules.

In a related embodiment, the present invention provides a method for treating a human patient, the method comprising the step of orally administering to the patient a nutritional supplement comprising, on a 24-hour dosage basis: (a) about 25,000 IU to about 60,000 IU of Vitamin A, (b) about 420 mg to about 540 mg of Vitamin C, (c) about 390 IU to about 840 IU of Vitamin E, (d) less than about 0.75 mg of zinc, (e) less than about 0.1 mg of copper, and (f) about 5 mg to about 35 mg of at least one carotenoid, wherein the at least one carotenoid is not β-carotene, and wherein the nutritional supplement is provided in the form of one or more capsules.

A fourth embodiment of the present invention provides a nutritional supplement comprising, on a 24-hour dosage basis: (a) about 500% to about 1200% of the RDA of Vitamin A, (b) about 700% to about 900% of the RDA of Vitamin C, (c) about 1300% to about 2800% of the RDA of Vitamin E, (d) about 45% to about 650% of the RDA of zinc, (e) about 35% to about 60% of the RDA of copper, and (f) about 5 mg to about 35 mg of at least one carotenoid, wherein the at least one carotenoid is not β-carotene, and wherein the nutritional supplement is provided in the form of one or more capsutes.

Another embodiment of the present invention provides a nutritional supplement comprising, on a 24-hour dosage basis: (a) about 25,000 IU to about 60,000 IU of Vitamin A,(b) about 420 mg to about 540 mg of Vitamin C,(c) about 390 mg to about 840 IU of Vitamin E, (d) about 7 mg to about 97.5 mg of zinc, (e) about 0.7 mg to about 1.2 mg of copper, and (f) about 5 mg to about 35 mg of at least one carotenoid, wherein the at least one carotenoid is not β-carotene, and wherein the nutritional supplement is provided in the form of one or more capsules.

In yet another embodiment, the present invention provides a method for treating a human patient, the method comprising the step of orally administering to the patient a nutritional supplement comprising, on a 24-hour dosage basis: (a) about 25,000 IU to about 60,000 IU of Vitamin A, (b) about 420 mg to about 540 mg of Vitamin C, (c) about 390 IU to about 840 IU of Vitamin E, (d) about 7 mg to about 97.5 mg of zinc, (e) about 0.7 mg to about 1.2 mg of copper, and (f) about 5 mg to about 35 mg of at least one carotenoid, wherein the at least one carotenoid is not β-carotene, and wherein the nutritional supplement is provided in the form of one or more capsules.

### DETAILED DESCRIPTION OF THE INVENTION

The invention provides, in one embodiment, a nutritional supplement comprising, on a 24-hour dosage basis: (a) about 500% to about 1200% of the RDA of Vitamin A, (b) about 700% to about 900% of the RDA of Vitamin C, (c) about 1300% to about 2800% of the RDA of Vitamin E, (d) less than about 5% of the RDA of zinc, (e) less than about 5% of the RDA of copper, and (d) about 5 mg to about 35 mg of at least one carotenoid.

Vitamin A plays an important role in vision, bone growth, reproduction, cell division and cell differentiation. With respect to vision, Vitamin A has been shown to help maintain the surface linings of the eyes. Furthermore, certain proforms of Vitamin A (e.g., β-carotene) have been shown to have very high antioxidant potentials. Currently, the U.S. Recommended Dietary Allowance for Vitamin A is 5,000 International Units (IU) per day.

As noted above, one of the nutritional supplements of the invention comprises, on a 24-hour dosage basis, about 500% to about 1200%, preferably about 500% to about 600%, of the RDA of Vitamin A. In a preferred embodiment, the nutritional supplement comprises, on a 24-hour dosage basis, about 573% of the RDA of Vitamin A. The Vitamin A contained in the nutritional supplement of the invention may be provided by any suitable source including, but not limited to, retinol, β-carotene, and mixtures thereof. In order to ensure that a greater amount of the source of Vitamin A is more readily converted to Vitamin A upon administration of the nutritional supplement, the Vitamin A contained in the nutritional supplement of the invention preferably is provided as β-carotene.

Vitamin C, in the form of ascorbate, is found in the aqueous humor of human eyes. Vitamin C also possesses well known antioxidant properties. Because humans depend on external sources of vitamin C to meet their vitamin C requirements, supplementation of the dietary intake of Vitamin C has several benefits. For example, Vitamin C supplementation may help to ensure a high concentration of ascorbate in the aqueous humor of eyes via the active transport of ascorbate from the blood stream to the posterior chamber of the eyes. Currently, the RDA for Vitamin C is 60 mg per day.

In the first embodiment, the nutritional supplement of the invention comprises, on a 24-hour dosage basis, about 700% to about 900%, preferably about 700% to about 800%, of the RDA of Vitamin C. In a preferred embodiment, the nutritional supplement comprises, on a 24-hour dosage basis, about 753% of the RDA of Vitamin C. The Vitamin C contained in the nutritional supplement of the invention may be provided by any suitable source including, but not limited to, ascorbic acid, a conjugate base of ascorbic acid (e.g., sodium ascorbate), and mixtures thereof. Preferably, the Vitamin C contained in the nutritional supplement of the invention is provided as ascorbic acid.

Vitamin E is a fat-soluble vitamin that exists in eight different forms. Each form has its own biological activity, the measure of potency or functional use in the body. Vitamin E is a well-known antioxidant and may work synergistically with vitamin C in protecting vital cell function from normal oxidants. Once ingested, vitamin E is stored within the body and may contribute to the total body pool of vitamin E for up to one year. Currently, the RDA for Vitamin E is 30 IU per day.

The first embodiment of the nutritional supplement of the invention comprises, on a 24-hour dosage basis, about 1300% to about 2800%, preferably about 1300% to about 1400%, of the RDA of Vitamin E. In a preferred embodiment, the nutritional supplement comprises, on a 24-hour dosage basis, about 1333% of the RDA of Vitamin E. The Vitamin E contained in the nutritional supplement of the invention may be provided by any suitable source including, but not limited to, dl-α-tocopheryl acetate, trimethyl tocopheryl acetate, dl-α-tocopheryl succinate, and mixtures thereof. Preferably, the Vitamin E contained in the nutritional supplement of the invention is provided as dl-α-tocopheryl acetate.

Zinc is important in maintaining the health of an eye's retina and is an essential part of more than 100 enzymes involved in digestion, metabolism, reproduction, and wound healing. Furthermore, certain dosages of zinc have been shown to be significantly better than placebo in retarding macular degeneration changes. Currently, the RDA for zinc is 15 mg per day.

In the first embodiment of the present invention, however, the nutritional supplement of the invention may comprise, on a 24-hour dosage basis, less than about 5% of the RDA of zinc. In a preferred embodiment, the nutritional supplement of the invention comprises, on a 24-hour dosage basis, less than about 2%, more preferably less than about 1%, most preferably less than about 0.1%, of the RDA of zinc. In a particularly preferred embodiment, the nutritional supplement does not comprise a measurable amount of zinc.

Copper, like zinc, is another important cofactor for metalloenzymes, and is a second necessary cofactor for superoxide dismutase. Currently, the RDA for copper is 2 mg per day. The first embodiment of the nutritional supplement of the invention, however, may comprise, on a 24-hour dosage basis, less than about 5% of the RDA of copper. In a preferred embodiment, the nutritional supplement of the invention comprises, on a 24-hour dosage basis, less than about 2%, more preferably less than about 1%, and most preferably less than about 0. 1%, of the RDA of copper. In a particularly preferred embodiment, the nutritional supplement does not comprise a measurable amount of copper.

A nutritional supplement of the invention may further comprise, on a 24-hour dosage basis, about 5 mg to about 35 mg, preferably about 5 mg to about 7 mg, of at least one carotenoid. While there currently is no RDA for carotenoids themselves, certain carotenoids (e.g., lutein, zeaxanthin, etc.) are found in the retina of healthy eyes. Preferably, the at least one carotenoid contained in the nutritional supplement of the invention is not p-carotene. The at least one carotenoid may be any suitable carotenoid, including, but not limited to, α-carotene, β-cryptoxanthin, lutein, zeaxanthin, and mixtures thereof. Preferably, the at least one carotenoid is selected from the group consisting of lutein, zeaxanthin, and mixtures thereof. In a preferred embodiment, the at least one carotenoid is a mixture of about 5.5 mg to about 6.5 mg of lutein and about 200 μg to about 300 μg of zeaxanthin. In another preferred embodiment, the at least one carotenoid is a mixture of about 6 mg of lutein and about 260 μg of zeaxanthin.

As noted above, this embodiment of the nutritional supplement of the invention comprises Vitamin A, Vitamin C, Vitamin E, at least one carotenoid which is not β-carotene (e.g., lutein and/or zeaxanthin), and less than certain specified amounts of zinc and copper. In another embodiment of this nutritional supplement, the nutritional supplement preferably consists essentially of Vitamin A, Vitamin C, Vitamin E, at least one carotenoid which is not β-carotene (e.g., lutein and/or zeaxanthin), and less than the specified amounts of zinc and copper. As utilized herein, the term “consists essentially of” does not exclude from the nutritional supplement inactive ingredients that do not materially affect the basic and novel characteristics of the nutritional supplement of the invention. For example, a nutritional supplement according to the invention which consists essentially of Vitamin A, Vitamin C, Vitamin E, at least one carotenoid which is not β-carotene, and less than certain amounts of zinc and copper may also contain pharmaceutically-acceptable inactive ingredients including, but not limited to, gelatin, cellulose, silicon dioxide, and/or magnesium stearate.

In an alternative second embodiment, the invention provides a nutritional supplement that further comprises both zinc and copper in amounts approaching or exceeding their respective RDAs. In particular, the invention provides a nutritional supplement comprising, on a 24-hour dosage basis: (a) about 500% to about 1200% of the RDA of Vitamin A, (b) about 700% to about 900% of the RDA of Vitamin C, (c) about 1300% to about 2800% of the RDA of Vitamin E, (d) about 45% to about 650% of the RDA of zinc, (e) about 35% to about 60% of the RDA of copper, and (f) about 5 mg to about 35 mg of at least one carotenoid.

As noted above, the second embodiment of the nutritional supplement of the invention comprises, on a 24-hour dosage basis, about 45% to about 650% of the RDA of zinc. In a preferred embodiment, the nutritional supplement of the invention comprises, on a 24-hour dosage basis, about 400% to about 650%, more preferably about 433% to about 650%, most preferably about 464%, of the RDA of zinc. The zinc contained in the nutritional supplement of the invention may be provided by any suitable source, including, but not limited to, zinc oxide, zinc gluconate, zinc phosphate, zinc sulfate, and mixtures thereof. Preferably, the zinc contained in the nutritional supplement of the invention is provided as zinc oxide.

The second embodiment of the nutritional supplement of the invention comprises, on a 24-hour dosage basis, about 35% to about 60% of the RDA of copper. In a preferred embodiment, the nutritional supplement of the invention comprises, on a 24-hour dosage basis, about 40% of the RDA of copper. The copper contained in the nutritional supplement of the invention may be provided by any suitable source including, but not limited to, cupric oxide (i.e., copper (II) oxide), copper gluconate, and mixtures thereof. Preferably, the copper contained in the nutritional supplement of the invention is provided as cupric oxide.

The other characteristics of this embodiment of the nutritional supplement of the invention (e.g., sources of Vitamin A, Vitamin C, Vitamin E, and the at least one carotenoid, etc.) may be the same as set forth above for the first embodiment of the nutritional supplement of the invention.

As noted above, the second embodiment of the nutritional supplement of the invention preferably comprises Vitamin A, Vitamin C, Vitamin E, zinc, copper, and at least one carotenoid which is not β-carotene (e.g., lutein and/or zeaxanthin). In another embodiment of this nutritional supplement, the nutritional supplement preferably consists essentially of Vitamin A, Vitamin C, Vitamin E, zinc, copper, and at least one carotenoid which is not β-carotene (e.g., lutein and/or zeaxanthin). As utilized herein, the term “consists essentially of” does not exclude from the nutritional supplement inactive ingredients that do not materially affect the basic and novel characteristics of the nutritional supplement of the invention. For example, a nutritional supplement according to this embodiment of the invention which consists essentially of Vitamin A, Vitamin C, Vitamin E, zinc, copper, and at least one carotenoid which is not β-carotene may also contain pharmaceutically-acceptable inactive ingredients such as gelatin, cellulose, silicon dioxide, and/or magnesium stearate.

In a third embodiment, the invention further provides a nutritional supplement comprising, on a 24-hour dosage basis: (a) about 25,000 IU to about 60,000 IU of Vitamin A, (b) about 420 mg to about 540 mg of Vitamin C, (c) about 390 IU to about 840 IU of Vitamin E, (d) less than about 0.75 mg of zinc, (e) less than about 0.1 mg of copper, and (f) about 5 mg to about 35 mg of at least one carotenoid.

As noted above, the third embodiment of the nutritional supplement of the invention comprises, on a 24-hour dosage basis, about 25,000 IU to about 60,000 IU, preferably about 25,000 IU to about 30,000 IU, of Vitamin A. Preferably, this embodiment of the nutritional supplement comprises, on a 24-hour dosage basis, about 28,640 IU of Vitamin A. The Vitamin A contained in this embodiment of the nutritional supplement may be provided by any suitable source including, but not limited to, those sources set forth above for the first embodiment of the nutritional supplement of the invention. Preferably, the Vitamin A contained in the third embodiment of the nutritional supplement of the invention is provided as β-carotene.

The third embodiment of the nutritional supplement of the invention comprises, on a 24-hour dosage basis, about 420 mg to about 540 mg, preferably about 420 mg to about 480 mg, of Vitamin C. Preferably, this embodiment of the nutritional supplement comprises, on a 24-hour dosage basis, about 452 mg of Vitamin C. The Vitamin C contained in this embodiment of the nutritional supplement may be provided by any suitable source including, but not limited to, those sources set forth for the first embodiment of the nutritional supplement of the invention. Preferably, the Vitamin C contained in the third embodiment of the nutritional supplement of the invention is provided as ascorbic acid.

The third embodiment of the nutritional supplement of the invention comprises, on a 24-hour dosage basis, about 390 IU to about 840 IU, preferably about 390 IU to about 420 IU, of Vitamin E. Preferably, this embodiment of the nutritional supplement comprises about 400 IU of Vitamin E. The Vitamin E contained in this embodiment of the nutritional supplement may be provided by any suitable source including, but not limited to, those sources set forth for the first embodiment of the nutritional supplement of the invention. Preferably, the Vitamin E contained in the third embodiment of the nutritional supplement of the invention is provided as dl-α-tocopheryl acetate.

The third embodiment of the nutritional supplement of the invention comprises, on a 24-hour dosage basis, less than about 0.75 mg of zinc. In a preferred embodiment, the nutritional supplement of the invention comprises, on a 24-hour dosage basis, less than about 0.5 mg, more preferably less than about 0.1 mg, and most preferably less than about 0.01 mg, of zinc. In a particularly preferred embodiment, the nutritional supplement does not comprise a measurable amount of zinc.

The third embodiment of the nutritional supplement of the invention comprises, on a 24-hour dosage basis, less than about 0.1 mg of copper. In a preferred embodiment, the nutritional supplement of the invention comprises, on a 24-hour dosage basis, less than about 0.05 mg, more preferably less than about 0.01 mg, and most preferably less than about 0.001 mg, of copper. In a particularly preferred embodiment, the nutritional supplement does not comprise a measurable amount of copper.

The third embodiment of the nutritional supplement of the invention further comprises, on a 24-hour dosage basis, about 5 mg to about 35 mg, preferably about 5 mg to about 7 mg, of at least one carotenoid. Preferably, the at least one carotenoid contained in this embodiment of the nutritional supplement of the invention is not β-carotene. The at least one carotenoid may be any suitable carotenoid, including, but not limited to, those carotenoids set forth for the first embodiment of the nutritional supplement of the invention. Preferably, the at least one carotenoid is selected from the group consisting of lutein, zeaxanthin, and mixtures thereof. In a preferred embodiment, the at least one carotenoid is a mixture of about 5.5 mg to about 6.5 mg of lutein and about 200 μg to about 300 μg of zeaxanthin. In another preferred embodiment, the at least one carotenoid is a mixture of about 6 mg of lutein and about 260 μg of zeaxanthin.

As noted above, the third embodiment of the nutritional supplement of the invention comprises Vitamin A, Vitamin C, Vitamin E, at least one carotenoid which is not β-carotene (e.g., lutein and/or zeaxanthin), and less than certain specified amounts of zinc and copper. In another embodiment of this nutritional supplement, the nutritional supplement preferably consists essentially of Vitamin A, Vitamin C, Vitamin E, at least one carotenoid which is not β-carotene (e.g., lutein and/or zeaxanthin), and less than the specified amounts of zinc and copper. As utilized herein, the term “consists essentially of” does not exclude from the nutritional supplement inactive ingredients that do not materially affect the basic and novel characteristics of the nutritional supplement of the invention. For example, a nutritional supplement according to the invention which consists essentially of Vitamin A, Vitamin C, Vitamin E, at least one carotenoid which is not 1-carotene, and less than certain amounts of zinc and copper may also contain pharmaceutically-acceptable inactive ingredients such as gelatin, cellulose, silicon dioxide, and/or magnesium stearate.

The invention also provides an additional fourth embodiment of the preferred nutritional supplement comprising both zinc and copper in amounts approaching or greater than their respective RDAs. In particular, the invention provides a nutritional supplement comprising, on a 24-hour dosage basis: (a) about 25,000 IU to about 60,000 IU of Vitamin A, (b) about 420 mg to about 540 mg of Vitamin C, (c) about 390 IU to about 840 IU of Vitamin E, (d) about 7 mg to about 97.5 mg of zinc, (e) about 0.7 mg to about 1.2 mg of copper, and (f) about 5 mg to about 35 mg of at least one carotenoid.

As noted above, the fourth embodiment of the nutritional supplement of the invention comprises, on a 24-hour dosage basis, about 7 mg to about 97.5 mg of zinc. Preferably, this embodiment of the nutritional supplement of the invention comprises, on a 24-hour dosage basis, about 60 mg to about 97.5 mg, more preferably about 65 mg to about 97.5 mg, and most preferably about 69.6 mg, of zinc. The zinc contained in this embodiment of the nutritional supplement may be provided by any suitable source including, but not limited to, those sources set forth above for the second embodiment of the nutritional supplement of the invention. Preferably, the zinc contained in the nutritional supplement of the invention is provided as zinc oxide.

The fourth embodiment of the nutritional supplement of the invention also comprises, on a 24-hour dosage basis, about 0.7 mg to about 1.2 mg of copper. Preferably, this embodiment of the nutritional supplement of the invention comprises, on a 24-hour dosage basis, about 0.7 mg to about 0.9 mg, more preferably about 0.8 mg, of copper. The copper contained in this embodiment of the nutritional supplement may be provided by any suitable source including, but not limited to, those sources set forth for the second embodiment of the nutritional supplement of the invention. Preferably, the copper contained in the nutritional supplement of the invention is provided as cupric oxide.

The other characteristics of this embodiment of the nutritional supplement of the invention (e.g., sources of Vitamin A, Vitamin C, Vitamin E, and the at least one carotenoid, etc.) may be the same as set forth above for the third embodiment of the nutritional supplement of the invention.

As noted above, the fourth embodiment of the nutritional supplement of the invention comprises Vitamin A, Vitamin C, Vitamin E, zinc, copper, and at least one carotenoid which is not β-carotene (e.g., lutein and/or zeaxanthin). In another embodiment of this nutritional supplement, the nutritional supplement preferably consists essentially of Vitamin A, Vitamin C, Vitamin E, zinc, copper, and at least one carotenoid which is not β-carotene (e.g., lutein and/or zeaxanthin). As utilized herein, the term “consists essentially of” does not exclude from the nutritional supplement inactive ingredients that do not materially affect the basic and novel characteristics of the nutritional supplement of the invention. For example, a nutritional supplement according to the invention which consists essentially of Vitamin A, Vitamin C, Vitamin E, zinc, copper, and at least one carotenoid which is not β-carotene may also contain inactive ingredients such as gelatin, cellulose, silicon dioxide, and/or magnesium stearate.

The embodiments of the nutritional supplements of the invention described herein may further comprise other vitamins, minerals, suitable herbal or botanical extracts, and other suitable ingredients found in nutritional supplements. For example, additional vitamins suitable for inclusion in the nutritional supplement of the invention include, but are not limited to, Vitamin B₁, Vitamin B2, Vitamin B6, Vitamin B₁₂, Vitamin D, Vitamin K, niacin, folic acid, biotin, and pantothenic acid. The nutritional supplements may further comprise suitable amounts of an additional mineral or trace element including, but not limited to, calcium, iron, iodine, magnesium, selenium, manganese, chromium, potassium, phosphorous, boron, and molybdenum. Suitable herbal or botanical extracts include, but are not limited to, flaxseed oil, evening primrose oil, borage oil, marine lipid oil, grape seed extract (Vitis vinifera), rose hips (from, e.g., Rosa Canina, R. gallica, R. condita, or R. rugosa), broccoli (e.g., Brassica oleracea v. botyris), garlic (Allium sativum), barley grass (Hordeum vulgare), beet juice powder (Beta vulgaris), carrot powder (Daucus carota), papaya (Carrica papaya), pineapple extract (Ananas comosus), wheat grass (Triticum aestivum), apple pectin (Malus sylvestris), rutin (from, e.g., Sophora japonica, Fagopyrum esculetum, Nicotiana tabaccum, Forsythia suspensa, Hydrangea paniculata, Viola sp., or Eucalyptus macroryncha), parsley (Petroselinum crispum), hawthorn (Crataegus oxycantha), milk thistle extract (Silyburn marianum), curcuminoids (from, e.g., tumeric extract (Curcuma longa)), horsetail (Equisetum majus), alfalfa (Medicajo sativa), ginkgo biloba extract (Ginkgo biloba), kelp (Laminaria spp.), licorice root (Glycyrrhiza glabra), soy isoflavones (from, e.g., Glycine max), lycopene (from, e.g., tomato extract (Lycopersicum esculentum)), saw palmetto (Serenoa repens), and ginseng (Panax ginseng). The embodiments of the nutritional supplements of the invention described herein may also desirably contain chelating agents (e.g., clioquinol).

The above-described embodiments of the nutritional supplement of the invention may further comprise any suitable, pharmaceutically-acceptable inactive ingredients. Typically, inactive ingredients are included in the nutritional supplement of the invention in order to aid in providing the nutritional supplement in a capsule form or to aid in the absorption and/or uptake of the active ingredients (e.g., Vitamin A, Vitamin C, Vitamin E, zinc, copper, and at least one carotenoid) contained in the nutritional supplement. Suitable inactive ingredients include, but are not limited to binders, lubricants, and disintegrants, such as cellulose, gelatin, magnesium stearate, water, vegetable oil, glycerin, beeswax, and silicon dioxide (i.e., silica).

The 24-hour dosage of each embodiment of the nutritional supplement of the invention preferably is provided in the form of one or more capsules. The capsule(s) containing the nutritional supplement may be formed from any suitable material. Preferably, the capsule(s) containing the nutritional supplement of the invention are gelatin capsule(s). In a preferred embodiment, the 24-hour dosage of each embodiment of the nutritional supplement is provided in one capsule. In such an embodiment, the active ingredients of the nutritional supplement preferably comprise about 70% or more (e.g., about 75% or more) of the total weight of the capsule. In particular, for those embodiments of the nutritional supplement of the invention comprising Vitamin A, Vitamin C, Vitamin E, and at least one carotenoid (e.g., lutein and/or zeaxanthin), the Vitamin A, Vitamin C, Vitamin E, and at least one carotenoid preferably comprise about 70% or more (e.g., about 75% or more) of the total weight of the capsule. Alternatively, for those embodiments of the nutritional supplement of the invention comprising Vitamin A, Vitamin C, Vitamin E, zinc, copper, and at least one carotenoid (e.g., lutein and/or zeaxanthin), the Vitamin A, Vitamin C, Vitamin E, zinc, copper, and at least one carotenoid preferably comprise about 70% or more (e.g., about 75% or more) of the total weight of the capsule. In other embodiments, the 24-hour dosage of the nutritional supplement of the invention is provided in the form of four, more preferably two, capsules. In such embodiments, the active ingredients of the nutritional supplement may comprise any suitable fraction of the total weight of the capsule, but preferably comprise about 70% or more (e.g., about 75% or more) of the total weight of the capsule.

The invention further provides a method for the treatment of a human or other animal by administering to the human or animal at least one embodiment of the nutritional supplement described herein. While not wishing to be bound to any particular theory, it is believed that the method of the invention will strengthen and promote retinal health through the prevention, stabilization, reversal and/or treatment of visual acuity loss in people with particular ocular diseases (e.g., macular degeneration).

In a preferred embodiment of the method of the invention, the method comprises the step of orally administering to a patient a nutritional supplement comprising, on a 24-hour dosage basis: (a) about 25,000 IU to about 60,000 IU of Vitamin A, (b) about 420 to about 540 mg of Vitamin C, (c) about 390 IU to about 840 IU of Vitamin E, and (d) about 5 mg to about 35 mg of at least one carotenoid. In one embodiment, the nutritional supplement preferably comprises, on a 24-hour dosage basis, less than about 0.75 mg of zinc and less than about 0.1 mg of copper. In another preferred embodiment, the nutritional supplement further comprises, on a 24-hour dosage basis, about 7 mg to about 97.5 mg of zinc and about 0.7 mg to about 1.2 mg of copper. The other characteristics of the nutritional supplement (e.g., sources of Vitamin A, Vitamin C, Vitamin E, zinc, copper, and the at least one carotenoid, inactive ingredients, etc.) used in the method of the invention may be the same as set forth above for the third and fourth embodiments of the nutritional supplement of the invention.

In a more preferred embodiment of the above-described method of the invention, the nutritional supplement comprises, on a 24-hour dosage basis: (a) about 28,640 IU of Vitamin A as β-carotene, (b) about 452 mg of Vitamin C, (c) about 400 IU of Vitamin E as dl-α-tocopheryl acetate, (d) less than about 0.75 mg of zinc, (e) less than about 0.1 mg of copper, (f) about 6 mg of lutein, and (g) about 260 μg of zeaxanthin.

In another embodiment of the method of the invention, the 24-hour dosage of the nutritional supplement is provided in two capsules, each capsule comprising: (a) about 14,320 IU of Vitamin A as β-carotene, (b) about 226 mg of Vitamin C, (c) about 200 IU of Vitamin E as dl-α-tocopheryl acetate, (d) less than about 0.375 mg of zinc, (e) less than about 0.05 mg of copper, (f) about 3 mg of lutein, and (g) about 130 μg of zeaxanthin. In such an embodiment of the method of the invention, the method further comprises administering at least two capsules to the patient in one 24-hour period (e.g., per day), and the two capsules preferably are administered to the patient at two different times during the 24-hour period (e.g., in the morning and in the evening).

In another preferred embodiment of the above-described method of the invention, the nutritional supplement comprises, on a 24-hour dosage basis: (a) about 28,640 IU of Vitamin A as β-carotene, (b) about 452 mg of Vitamin C as ascorbic acid, (c) about 400 IU of Vitamin E as dl-α-tocopheryl acetate, (d) about 69.6 mg of zinc as zinc oxide, (e) about 0.8 mg of copper as cupric oxide, (f) about 6 mg of lutein, and (g) about 260 μg of zeaxanthin.

In another embodiment of the method of the invention, the 24-hour dosage of the nutritional supplement is provided in two capsules, each capsule comprising: (a) about 14,320 IU of Vitamin A as β-carotene, (b) about 226 mg of Vitamin C as ascorbic acid, (c) about 200 IU of Vitamin E as dl-α-tocopheryl acetate, (d) about 34.8 mg of zinc as zinc oxide, (e) about 0.4 mg of copper as cupric oxide, (f) about 3 mg of lutein, and (g) about 130 μg of zeaxanthin. In such an embodiment of the method of the invention, the method further comprises administering at least two capsules to the patient in one 24-hour period (e.g., per day), and the two capsules preferably are administered to the patient at two different times during the 24-hour period (e.g., in the morning and in the evening).

The following examples further illustrate the invention but, of course, should not be construed as in any way limiting its scope.

### EXAMPLE 1

This example demonstrates the preparation of a nutritional supplement according to the invention. The raw materials set forth in Table 1 are weighed and blended for approximately 10 minutes to obtain a homogeneous mixture. The amounts set forth in Table 1 for each of the raw materials have been calculated to include overages necessary to account for manufacturing losses and to attain the label claim for the nutritional supplement over the shelf-life of the nutritional supplement, which typically is about 3 years (i.e., about 36 months). Next, 477.5 mg of the resulting mixture is placed in a size 1 hard shell, gelatin capsule, which capsule is subsequently cleaned.

TABLE 1
Raw Materials for the Nutritional Supplement.

| Raw Material | Amount |
|---|---|
| Vitamin A (as β-carotene) | 30.0 g |
| Vitamin C (as ascorbic acid) | 130.0 g |
| Vitamin E (as dl-α-tocopheryl acetate) | 146.7 g |
| Lutein/Zeaxanthin mixture | 34.5 g |
| Gelatin | 80.0 g |
| Cellulose | 48.3 g |
| Silicon Dioxide | 4.0 g |
| Magnesium Stearate | 4.0 g |

Two capsules containing the resulting nutritional supplement are intended to be taken twice daily, for a total of four capsules per 24-hour period. The label claim for each active ingredient contained in the capsule, as well as the label claim per 24-hour dosage and label claim per 24-hour dosage expressed as a percent of the RDA, is set forth in Table 2.

TABLE 2
Active Ingredient Label Claim per Capsule, per 24-hour
Period, and Percent of RDA per 24-hour Dosage.

| | | Amount | Percent of RDA |
|---|---|---|---|
| | Amount per | per 24- | per 24-hour |
| Active Ingredient | Capsule | hour Dosage | Dosage |
| Vitamin A (as β- | 4,296 μg | 17,184 μg | 573% |
| carotene) | (7,160 IU) | (28,640 IU) | |
| Vitamin C (as | 113 mg | 452 mg | 753% |
| ascorbic acid) | | | |
| Vitamin E (as dl-α- | 66.67 mg | 266.67 mg | 1333% |
| tocopheryl acetate) | (100 IU) | (400 IU) | |
| Lutein | 1.5 mg | 6 mg | — |
| Zeaxanthin | 65 μg | 260 μg | — |

### EXAMPLE 2

This example demonstrates the preparation of a nutritional supplement according to the invention. The raw materials set forth in Table 3 are weighed and blended for approximately 10 minutes to obtain a homogeneous mixture. The amounts set forth in Table 3 for each of the raw materials have been calculated to include overages necessary to account for manufacturing losses and to attain the label claim for the nutritional supplement over the shelf-life of the nutritional supplement, which typically is about 3 years (i.e., about 36 months). Next, 480.6 mg of the resulting mixture is placed in a size 1 hard shell, gelatin capsule, which capsule is subsequently cleaned.

TABLE 3
Raw Materials for the Nutritional Supplement.

| Raw Material | Amount | |
|---|---|---|
| Vitamin A (as β-carotene) | 30.0 | g |
| Vitamin C (as ascorbic acid) | 130.0 | g |
| Vitamin E (as dl-α-tocopheryl acetate) | 146.7 | g |
| Zinc (as zinc oxide) | 2.8 | g |
| Copper (as cupric oxide) | 300 | mg |
| Lutein/Zeaxanthin mixture | 34.5 | g |
| Gelatin | 80.0 | g |
| Cellulose | 48.3 | g |
| Silicon Dioxide | 4.0 | g |
| Magnesium Stearate | 4.0 | g |

Two capsules containing the resulting nutritional supplement are intended to be taken twice daily, for a total of four capsules per 24-hour period. The label claim for each active ingredient contained in the capsule, as well as the label claim per 24-hour dosage and label claim per 24-hour dosage expressed as a percent of the RDA, is set forth in Table 4.

TABLE 4
Active Ingredient Label Claim per Capsule, per 24-hour
Period, and Percent of RDA per 24-hour Dosage.

| | | | Percent of RDA |
|---|---|---|---|
| | Amount per | Amount per 24- | per 24-hour |
| Active Ingredient | Capsule | hour Dosage | Dosage |
| Vitamin A (as β- | 4,296 μg | 17,184 μg | 573% |
| carotene) | (7,160 IU) | (28,640 IU) | |
| Vitamin C (as | 113 mg | 452 mg | 753% |
| ascorbic acid) | | | |
| Vitamin E (as dl-α- | 66.67 mg | 266.67 mg | 1333% |
| tocopheryl acetate) | (100 IU) | (400 IU) | |
| Zinc (as zinc oxide) | 2 mg | 8 mg | 53.3% |
| Copper (as cupric | 0.2 mg | 0.8 mg | 40% |
| oxide) | | | |
| Lutein | 1.5 mg | 6 mg | — |
| Zeaxanthin | 65 μg | 260 μg | — |

### EXAMPLE 3

This example demonstrates the preparation of a nutritional supplement according to the invention. The raw materials set forth in Table 5 are weighed and blended for approximately 10 minutes to obtain a homogeneous mixture. The amounts set forth in Table 5 for each of the raw materials have been calculated to include overages necessary to account for manufacturing losses and to attain the label claim for the nutritional supplement over the shelf-life of the nutritional supplement, which typically is about 3 years (i.e., about 36 months). Next, 501.7 mg of the resulting mixture is placed in a size 1 hard shell, gelatin capsule, which capsule is subsequently cleaned.

TABLE 5
Raw Materials for the Nutritional Supplement.

| Raw Material | Amount | |
|---|---|---|
| Vitamin A (as β-carotene) | 30.0 | g |
| Vitamin C (as ascorbic acid) | 130.0 | g |
| Vitamin E (as dl-α-tocopheryl acetate) | 146.7 | g |
| Zinc (as zinc oxide) | 23.93 | g |
| Copper (as cupric oxide) | 300 | mg |
| Lutein/Zeaxanthin mixture | 34.5 | g |
| Gelatin | 80.0 | g |
| Cellulose | 48.3 | g |
| Silicon Dioxide | 4.0 | g |
| Magnesium Stearate | 4.0 | g |

Two capsules containing the resulting nutritional supplement are intended to be taken twice daily, for a total of four capsules per 24-hour period. The label claim for each active ingredient contained in the capsule, as well as the label claim per 24-hour dosage and label claim per 24-hour dosage expressed as a percent of the RDA, is set forth in Table 6.

TABLE 6
Active Ingredient Label Claim per Capsule, per 24-hour
Period, and Percent of RDA per 24-hour Dosage.

| | | | Percent of RDA |
|---|---|---|---|
| | Amount per | Amount per 24- | per 24-hour |
| Active Ingredient | Capsule | hour Dosage | Dosage |
| Vitamin A (as β- | 4,296 μg | 17,184 μg | 573% |
| carotene) | (7,160 IU) | (28,640 IU) | |
| Vitamin C (as | 113 mg | 452 mg | 753% |
| ascorbic acid) | | | |
| Vitamin E (as dl-α- | 66.67 mg | 266.67 mg | 1333% |
| tocopheryl acetate) | (100 IU) | (400 IU) | |
| Zinc (as zinc oxide) | 17.4 mg | 69.6 mg | 464% |
| Copper (as cupric | 0.2 mg | 0.8 mg | 40% |
| oxide) | | | |
| Lutein | 1.5 mg | 6 mg | — |
| Zeaxanthin | 65 μg | 260 μg | — |

### EXAMPLE 4

This example demonstrates the preparation of a nutritional supplement according to the invention. The raw materials set forth in Table 7 are weighed and blended for approximately 10 minutes to obtain a homogeneous mixture. The amounts set forth in Table 7 for each of the raw materials have been calculated to include overages necessary to account for manufacturing losses and to attain the label claim for the nutritional supplement over the shelf-life of the nutritional supplement, which typically is about 3 years (i.e., about 36 months). Next, 955 mg of the resulting mixture is placed in a size 00 hard shell, gelatin capsule, which capsule is subsequently cleaned.

TABLE 7
Raw Materials for the Nutritional Supplement.

| Raw Material | Amount |
|---|---|
| Vitamin A (as β-carotene) | 30.0 g |
| Vitamin C (as ascorbic acid) | 130.0 g |
| Vitamin E (as dl-α-tocopheryl acetate) | 146.7 g |
| Lutein/Zeaxanthin mixture | 34.5 g |
| Gelatin | 80.0 g |
| Cellulose | 48.3 g |
| Silicon Dioxide | 4.0 g |
| Magnesium Stearate | 4.0 g |

One capsule containing the resulting nutritional supplement is intended to be taken twice daily, for a total of two capsules per 24-hour period. The label claim for each active ingredient contained in the capsule, as well as the label claim per 24-hour dosage and label claim per 24-hour dosage expressed as a percent of the RDA, is set forth in Table 8.

TABLE 8
Active Ingredient Label Claim per Capsule, per 24-hour
Period, and Percent of RDA per 24-hour Dosage.

| | | | Percent of RDA |
|---|---|---|---|
| | Amount per | Amount per 24- | per 24-hour |
| Active Ingredient | Capsule | hour Dosage | Dosage |
| Vitamin A (as β- | 8,592 μg | 17,184 μg | 573% |
| carotene) | (14,320 IU) | (28,640 IU) | |
| Vitamin C (as | 226 mg | 452 mg | 753% |
| ascorbic acid) | | | |
| Vitamin E (as dl-α- | 133.34 mg | 266.67 mg | 1333% |
| tocopheryl acetate) | (200 IU) | (400 IU) | |
| Lutein | 3 mg | 6 mg | — |
| Zeaxanthin | 130 μg | 260 μg | — |

### EXAMPLE 5

This example demonstrates the preparation of a nutritional supplement according to the invention. The raw materials set forth in Table 9 are weighed and blended for approximately 10 minutes to obtain a homogeneous mixture. The amounts set forth in Table 9 for each of the raw materials have been calculated to include overages necessary to account for manufacturing losses and to attain the label claim for the nutritional supplement over the shelf-life of the nutritional supplement, which typically is about 3 years (i.e., about 36 months). Next, 961.2 mg of the resulting mixture is placed in a size 00 hard shell, gelatin capsule, which capsule is subsequently cleaned.

TABLE 9
Raw Materials for the Nutritional Supplement.

| Raw Material | Amount | |
|---|---|---|
| Vitamin A (as β-carotene) | 30.0 | g |
| Vitamin C (as ascorbic acid) | 130.0 | g |
| Vitamin E (as dl-α-tocopheryl acetate) | 146.7 | g |
| Zinc (as zinc oxide) | 2.8 | g |
| Copper (as cupric oxide) | 300 | mg |
| Lutein/Zeaxanthin mixture | 34.5 | g |
| Gelatin | 80.0 | g |
| Cellulose | 48.3 | g |
| Silicon Dioxide | 4.0 | g |
| Magnesium Stearate | 4.0 | g |

One capsule containing the resulting nutritional supplement is intended to be taken twice daily, for a total of two capsules per 24-hour period. The label claim for each active ingredient contained in the capsule, as well as the label claim per 24-hour dosage and label claim per 24-hour dosage expressed as a percent of the RDA, is set forth in Table 10.

TABLE 10
Active Ingredient Label Claim per Capsule, per 24-hour
Period, and Percent of RDA per 24-hour Dosage.

| | | | Percent of RDA |
|---|---|---|---|
| | Amount per | Amount per 24- | per 24-hour |
| Active Ingredient | Capsule | hour Dosage | Dosage |
| Vitamin A (as β- | 8,592 μg | 17,184 μg | 573% |
| carotene) | (14,320 IU) | (28,640 IU) | |
| Vitamin C (as | 226 mg | 452 mg | 753% |
| ascorbic acid) | | | |
| Vitamin E (as dl-α- | 133.34 mg | 266.67 mg | 1333% |
| tocopheryl acetate) | (200 IU) | (400 IU) | |
| Zinc (as zinc oxide) | 4 mg | 8 mg | 53.3% |
| Copper (as cupric | 0.4 mg | 0.8 mg | 40% |
| oxide) | | | |
| Lutein | 3 mg | 6 mg | — |
| Zeaxanthin | 130 μg | 260 μg | — |

### EXAMPLE 6

This example demonstrates the preparation of a nutritional supplement according to the invention. The raw materials set forth in Table 11 are weighed and blended for approximately 10 minutes to obtain a homogeneous mixture. The amounts set forth in Table 11 for each of the raw materials have been calculated to include overages necessary to account for manufacturing losses and to attain the label claim for the nutritional supplement over the shelf-life of the nutritional supplement, which typically is about 3 years (i.e., about 36 months). Next, 1003.4 mg of the resulting mixture is placed in a size 00 hard shell, gelatin capsule, which capsule is subsequently cleaned.

TABLE 11
Raw Materials for the Nutritional Supplement.

| Raw Material | Amount | |
|---|---|---|
| Vitamin A (as β-carotene) | 30.0 | g |
| Vitamin C (as ascorbic acid) | 130.0 | g |
| Vitamin E (as dl-α-tocopheryl acetate) | 146.7 | g |
| Zinc (as zinc oxide) | 23.93 | g |
| Copper (as cupric oxide) | 300 | mg |
| Lutein/Zeaxanthin mixture | 34.5 | g |
| Gelatin | 80.0 | g |
| Cellulose | 48.3 | g |
| Silicon Dioxide | 4.0 | g |
| Magnesium Stearate | 4.0 | g |

One capsule containing the resulting nutritional supplement is intended to be taken twice daily, for a total of two capsules per 24-hour period. The label claim for each active ingredient contained in the capsule, as well as the label claim per 24-hour dosage and label claim per 24-hour dosage expressed as a percent of the RDA, is set forth in Table 12.

TABLE 12
Active Ingredient Label Claim per Capsule, per 24-hour Period, and Percent of
RDA per 24-hour Dosage.

| | | | | | Percent of RDA |
|---|---|---|---|---|---|
| | Amount per | | Amount per 24- | | per 24-hour |
| Active Ingredient | Capsule | | hour Dosage | | Dosage |
| Vitamin A (as β- | 8,592 | μg (14,320 IU) | 17,184 | μg (28,640 IU) | 573% |
| carotene) | | | | | |
| Vitamin C (as | 226 | mg | 452 | mg | 753% |
| ascorbic acid) | | | | | |
| Vitamin E (as dl-α- | 133.34 | mg (200 IU) | 266.67 | mg (400 IU) | 1333% |
| tocopheryl acetate) | | | | | |
| Zinc (as zinc oxide) | 34.8 | mg | 69.6 | mg | 464% |
| Copper (as cupric | 0.4 | mg | 0.8 | mg | 40% |
| oxide) | | | | | |
| Lutein | 3 | mg | 6 | mg | — |
| Zeaxanthin | 130 | μg | 260 | μg | — |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nutritional supplement comprising, on a 24-hour dosage basis:
(a) about 500% to about 1200% of the RDA of Vitamin A,
(b) about 700% to about 900% of the RDA of Vitamin C,
(c) about 1300% to about 2800% of the RDA of Vitamin E,
(d) less than about 5% of the RDA of zinc,
(e) less than about 5% of the RDA of copper, and
(f) about 5 mg to about 35 mg of at least one carotenoid,
wherein the at least one carotenoid is not β-carotene, and wherein the nutritional supplement is provided in the form of one or more capsules.

2. The nutritional supplement of claim 1, wherein the at least one carotenoid is selected from the group consisting of lutein, zeaxanthin, and mixtures thereof.

3. The nutritional supplement of claim 2, wherein the at least one carotenoid is a mixture of lutein and zeaxanthin.

4. The nutritional supplement of claim 3, wherein the at least one carotenoid is a mixture of about 5.5 mg to about 6.5 mg of lutein and about 200 μg to about 300 μg of zeaxanthin.

5. The nutritional supplement of claim 4, wherein the nutritional supplement comprises:
(a) about 573% of the RDA of Vitamin A as β-carotene,
(b) about 753% of the RDA of Vitamin C as ascorbic acid,
(c) about 1333% of the RDA of Vitamin E as dl-α-tocopheryl acetate,
(d) less than about 5% of the RDA of zinc,
(e) less than about 5% of the RDA of copper,
(f) about 6 mg of lutein, and
(g) about 260 μg of zeaxanthin.

6. A nutritional supplement comprising, on a 24-hour dosage basis:
(a) about 25,000 IU to about 60,000 IU of Vitamin A,
(b) about 420 mg to about 540 mg of Vitamin C,
(c) about 390 mg to about 840 IU of Vitamin E,
(d) less than about 0.75 mg of zinc,
(e) less than about 0.1 mg of copper,
(f) about 5 mg to about 35 mg of at least one carotenoid,
wherein the at least one carotenoid is not P-carotene, and wherein the nutritional supplement is provided in the form of one or more capsules.

7. The nutritional supplement of claim 6, wherein the at least one carotenoid is selected from the group consisting of lutein, zeaxanthin, and mixtures thereof.

8. The nutritional supplement of claim 7, wherein the at least one carotenoid is a mixture of lutein and zeaxanthin.

9. The nutritional supplement of claim 8, wherein the at least one carotenoid is a mixture of about 5.5 mg to about 6.5 mg of lutein and about 200 μg to about 300 μg of zeaxanthin.

10. The nutritional supplement of claim 9, wherein the nutritional supplement comprising, on a 24-hour dosage basis:
(a) about 28,640 IU of Vitamin A as β-carotene,
(b) about 452 mg of Vitamin C as ascorbic acid,
(c) about 400 IU of Vitamin E as dl-α-tocopheryl acetate,
(d) less than about 0.75 mg of zinc,
(e) less than about 0.1 mg of copper,
(f) about 6 mg of lutein, and
(g) about 260 μg of zeaxanthin.

11. A method for treating a human patient, the method comprising the step of orally administering to the patient a nutritional supplement comprising, on a 24-hour dosage basis:
(a) about 25,000 IU to about 60,000 IU of Vitamin A,
(b) about 420 mg to about 540 mg of Vitamin C,
(c) about 390 IU to about 840 IU of Vitamin E, and
(d) less than about 0.75 mg of zinc,
(e) less than about 0.1 mg of copper,
(f) about 5 mg to about 35 mg of at least one carotenoid,
wherein the at least one carotenoid is not β-carotene, and wherein the nutritional supplement is provided in the form of one or more capsules.

12. The method of claim 11, wherein the at least one carotenoid is selected from the group consisting of lutein, zeaxanthin, and mixtures thereof.

13. The method of claim 12, wherein the at least one carotenoid is a mixture of lutein and zeaxanthin.

14. The method of claim 13, wherein the at least one carotenoid is a mixture of about 5.5 mg to about 6.5 mg of lutein and about 200 μg to about 300 μg of zeaxanthin.

15. The method of claim 14, wherein the nutritional supplement comprises, on a 24-hour dosage basis:
(a) about 28,640 IU of Vitamin A as β-carotene,
(b) about 452 mg of Vitamin C as ascorbic acid,
(c) about 400 IU of Vitamin E as dl-α-tocopheryl acetate,
(d) less than about 0.75 mg of zinc,
(e) less than about 0.1 mg of copper,
(f) about 6 mg of lutein, and
(g) about 260 μg of zeaxanthin.

16. The method of claim 15, wherein the nutritional supplement is provided in two capsules, each capsule comprising:
(a) about 14,320 IU of Vitamin A as β-carotene,
(b) about 226 mg of Vitamin C as ascorbic acid,
(c) about 200 IU of Vitamin E as dl-α-tocopheryl acetate,
(d) less than about 0.375 mg of zinc,
(e) less than about 0.05 mg of copper,
(f) about 3 mg of lutein, and
(g) about 130 μg of zeaxanthin,
and wherein the method further comprises administering at least two capsules to the patient in one 24-hour period.

17. The method of claim 16, wherein the at least two capsules are administered to the patient at two different times during the 24-hour period.

18. A nutritional supplement comprising, on a 24-hour dosage basis:
(a) about 500% to about 1200% of the RDA of Vitamin A,
(b) about 700% to about 900% of the RDA of Vitamin C,
(c) about 1300% to about 2800% of the RDA of Vitamin E,
(d) about 45% to about 650% of the RDA of zinc,
(e) about 35% to about 60% of the RDA of copper, and
(f) about 5 mg to about 35 mg of at least one carotenoid,
wherein the at least one carotenoid is not β-carotene, and wherein the nutritional supplement is provided in the form of one or more capsules.

19. The nutritional supplement of claim 18, wherein the at least one carotenoid is selected from the group consisting of lutein, zeaxanthin, and mixtures thereof.

20. The nutritional supplement of claim 19, wherein the at least one carotenoid is a mixture of lutein and zeaxanthin.

21. The nutritional supplement of claim 20, wherein the at least one carotenoid is a mixture of about 5.5 mg to about 6.5 mg of lutein and about 200 μg to about 300 μg of zeaxanthin.

22. The nutritional supplement of claim 21, wherein the nutritional supplement comprises:
(a) about 573% of the RDA of Vitamin A as β-carotene,
(b) about 753% of the RDA of Vitamin C as ascorbic acid,
(c) about 1333% of the RDA of Vitamin E as dl-α-tocopheryl acetate,
(d) about 464% of the RDA of zinc as zinc oxide,
(e) about 40% of the RDA of copper as cupric oxide,
(f) about 6 mg of lutein, and
(g) about 260 μg of zeaxanthin.

23. A nutritional supplement comprising, on a 24-hour dosage basis:
(a) about 25,000 IU to about 60,000 IU of Vitamin A,
(b) about 420 mg to about 540 mg of Vitamin C,
(c) about 390 mg to about 840 IU of Vitamin E,
(d) about 7 mg to about 97.5 mg of zinc,
(e) about 0.7 mg to about 1.2 mg of copper, and
(f) about 5 mg to about 35 mg of at least one carotenoid,
wherein the at least one carotenoid is not β-carotene, and wherein the nutritional supplement is provided in the form of one or more capsules.

24. The nutritional supplement of claim 23, wherein the at least one carotenoid is selected from the group consisting of lutein, zeaxanthin, and mixtures thereof.

25. The nutritional supplement of claim 24, wherein the at least one carotenoid is a mixture of lutein and zeaxanthin.

26. The nutritional supplement of claim 25, wherein the at least one carotenoid is a mixture of about 5.5 mg to about 6.5 mg of lutein and about 200 μg to about 300 μg of zeaxanthin.

27. The nutritional supplement of claim 26, wherein the nutritional supplement comprises, on a 24-hour dosage basis:
(a) about 28,640 IU of Vitamin A as β-carotene,
(b) about 452 mg of Vitamin C as ascorbic acid,
(c) about 400 IU of Vitamin E as dl-α-tocopheryl acetate,
(d) about 69.6 mg of zinc as zinc oxide,
(e) about 0.8 mg of copper as cupric oxide,
(f) about 6 mg of lutein, and
(g) about 260 μg of zeaxanthin.

28. A method for treating a human patient, the method comprising the step of orally administering to the patient a nutritional supplement comprising, on a 24-hour dosage basis:
(a) about 25,000 IU to about 60,000 IU of Vitamin A,
(b) about 420 mg to about 540 mg of Vitamin C,
(c) about 390 IU to about 840 IU of Vitamin E,
(d) about 7 mg to about 97.5 mg of zinc,
(e) about 0.7 mg to about 1.2 mg of copper, and
(f) about 5 mg to about 35 mg of at least one carotenoid,
wherein the at least one carotenoid is not β-carotene, and wherein the nutritional supplement is provided in the form of one or more capsules.

29. The method of claim 28, wherein the at least one carotenoid is selected from the group consisting of lutein, zeaxanthin, and mixtures thereof.

30. The method of claim 29, wherein the at least one carotenoid is a mixture of lutein and zeaxanthin.

31. The method of claim 30, wherein the at least one carotenoid is a mixture of about 5.5 mg to about 6.5 mg of lutein and about 200 μg to about 300 μg of zeaxanthin.

32. The method of claim 31, wherein the nutritional supplement comprises, on a 24-hour dosage basis:
(a) about 28,640 IU of Vitamin A as ≢2-carotene,
(b) about 452 mg of Vitamin C as ascorbic acid,
(c) about 400 IU of Vitamin E as dl-α-tocopheryl acetate,
(d) about 69.6 mg of zinc as zinc oxide,
(e) about 0.8 mg of copper as cupric oxide,
(f) about 6 mg of lutein, and
(g) about 260 μg of zeaxanthin.

33. The method of claim 32, wherein the nutritional supplement is provided in two capsules, each capsule comprising:
(a) about 14,320 IU of Vitamin A as β-carotene,
(b) about 226 mg of Vitamin C as ascorbic acid,
(c) about 200 IU of Vitamin E as dl-α-tocopheryl acetate,
(d) about 34.8 mg of zinc as zinc oxide,
(e) about 0.4 mg of copper as cupric oxide,
(f) about 3 mg of lutein, and
(g) about 130 μg of zeaxanthin,
and wherein the method further comprises administering at least two capsules to the patient in one 24-hour period.

34. The method of claim 33, wherein the at least two capsules are administered to the patient at two different times during the 24-hour period.
